(19) United States
(12) Patent Application Publication
(10) Pub. No.: US 2014/0186931 A1
(43) Pub. Date: Jul. 3, 2014

(54) Process of Operating a Plurality of Photobioreactors

(71) Applicant: Pond Biofuels Inc., Scarborough (CA)

(72) Inventors: Jaime A. Gonzalez, Richmond Hill (CA); Steven C. Martin, Toronto (CA); Emidio Di Pietro, Brampton (CA); Tony Di Pietro, Brampton (CA); Max Kolesnik, Toronto (CA)

(73) Assignee: Pond Biofuels Inc., Scarborough (CA)

(21) Appl. No.: 14/089,278

(22) Filed: Nov. 25, 2013

Related U.S. Application Data
(63) Continuation of application No. 13/659,714, filed on Oct. 24, 2012, now abandoned.

(51) Int. Cl. C12N 1/12

(52) U.S. Cl. CPC C12N 1/12
USPC 435/257.1

(57) ABSTRACT

There is provided a process of operating a plurality of photobioreactors, comprising: while a carbon dioxide-comprising gaseous exhaust material producing process is effecting production of the carbon dioxide-comprising gaseous exhaust material, supplying at least a fraction of the produced carbon dioxide-comprising gaseous exhaust material to a respective reaction zone of each one of the phototobioreactors, in succession, wherein the at least a fraction of the produced carbon dioxide-comprising gaseous exhaust material being supplied defines a carbon dioxide-comprising gaseous exhaust supply.

### FIELD

The present disclosure relates to a process for growing biomass.

### BACKGROUND

The cultivation of phototrophic organisms has been widely practised for purposes of producing a fuel source. Exhaust gases from industrial processes have also been used to promote the growth of phototrophic organisms by supplying carbon dioxide for consumption by phototrophic organisms during photosynthesis. By providing exhaust gases for such purpose, environmental impact is reduced and, in parallel a potentially useful fuel source is produced. Challenges remain, however, to render this approach more economically attractive for incorporation within existing facilities.

### SUMMARY

In one aspect, there is provided a process of operating a plurality of photobioreactors, comprising: while a carbon dioxide-comprising gaseous exhaust material producing process is effecting production of the carbon dioxide-comprising gaseous exhaust material, supplying at least a fraction of the produced carbon dioxide-comprising gaseous exhaust material to a respective reaction zone of each one of the phototobioreactors, in succession, wherein the at least a fraction of the produced carbon dioxide-comprising gaseous exhaust material being supplied defines a carbon dioxide-comprising gaseous exhaust supply.

In another aspect, a process of operating a plurality of photobioreactors, comprising: while a carbon dioxide-comprising gaseous exhaust material producing process is effecting production of carbon dioxide-comprising gaseous exhaust material, and a carbon dioxide-comprising gaseous exhaust material supply, including at least a fraction of the produced carbon dioxide-comprising gaseous exhaust material, is supplied to a respective reaction zone of one or more of the photobioreactors to thereby define one or more supplied photobioreactors, after the pH, within the reaction zone, of any one of the one or more supplied photobioreactors, becomes disposed below a predetermined low pH limit, such that a low pH-disposed photobioreactor is defined, at least a fraction of the carbon dioxide-comprising gaseous exhaust material supply, being supplied to the low pH-disposed photobioreactors, is diverted to a respective reaction zone of each one of at least another one of the photobioreactors, for effecting supply of the diverted carbon dioxide-comprising gaseous exhaust material supply to the respective reaction zone of each one of the at least another one of the photobioreactors.

In a further aspect, there is provided a process of operating a plurality of photobioreactors, comprising: while a carbon dioxide-comprising gaseous exhaust material producing process is effecting production of carbon dioxide-comprising gaseous exhaust material, and a carbon dioxide-comprising gaseous exhaust material supply, including at least a fraction of the produced carbon dioxide-comprising gaseous exhaust material, is supplied to a respective reaction zone of one or more photobioreactors to thereby define one or more supplied photobioreactors, after the pH, within the reaction zone, of any one of the one or more supplied photobioreactors, becomes disposed above a predetermined maximum pH limit, such that a high pH-disposed photobioreactor is defined, at least a fraction of the carbon dioxide-comprising gaseous exhaust material supply being supplied to the respective reaction zone of each one of at least another one of the photobioreactors, whose reaction zone includes a pH that is less than the pH within the reaction zone of the high pH-disposed photobioreactor, is diverted to the high pH-disposed photobioreactor, for effecting supply of the diverted carbon dioxide-comprising gaseous exhaust material supply to the reaction zone of the high pH-disposed photobioreactor.

### BRIEF DESCRIPTION OF THE DRAWINGS

The process of the preferred embodiments of the invention will now be described with the following accompanying drawing:

FIG. 1 is a process flow diagram of an embodiment of the process.

### DETAILED DESCRIPTION

Reference throughout the specification to “some embodiments” means that a particular feature, structure, or characteristic described in connection with some embodiments are not necessarily referring to the same embodiments. Furthermore, the particular features, structure, or characteristics may be combined in any suitable manner with one another.

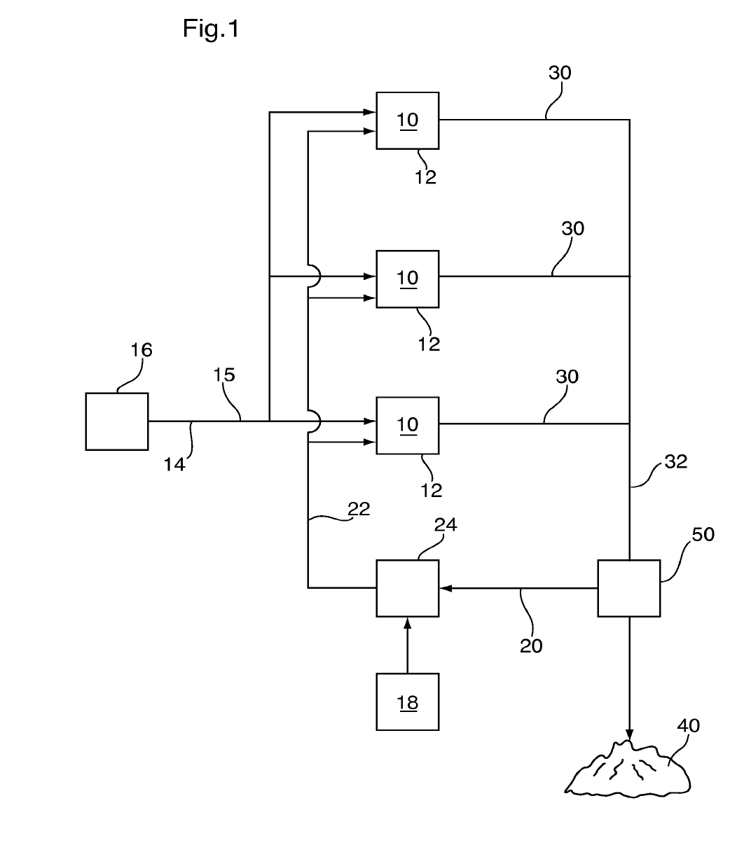

Referring to FIG. 1, there is provided a process of growing a phototrophic biomass within a plurality of photobioreactors 12. Each one of the photobioreactors includes a respective reaction zone 10.

The reaction zone 10 includes a reaction mixture that is operative for effecting photosynthesis upon exposure to photosynthetically active light radiation. The reaction mixture includes phototrophic biomass, carbon dioxide, and water. In some embodiments, the reaction zone includes phototrophic biomass and carbon dioxide disposed in an aqueous medium. Within the reaction zone 10, the phototrophic biomass is disposed in mass transfer communication with both of carbon dioxide and water.

“Phototrophic organism” is an organism capable of phototrophic growth in the aqueous medium upon receiving light energy, such as plant cells and micro-organisms. The phototrophic organism is unicellular or multicellular. In some embodiments, for example, the phototrophic organism is an organism which has been modified artificially or by gene manipulation. In some embodiments, for example, the phototrophic organism is an algae. In some embodiments, for example, the algae is microalgae.

“Phototrophic biomass” is at least one phototrophic organism. In some embodiments, for example, the phototrophic biomass includes more than one species of phototrophic organisms.

“Reaction zone 10” defines a space within which the growing of the phototrophic biomass is effected. In some embodiments, for example, pressure within the reaction zone is atmospheric pressure.

“Photobioreactor 12” is any structure, arrangement, land formation or area that provides a suitable environment for the growth of phototrophic biomass. Examples of specific structures which can be used is a photobioreactor 12 by providing space for growth of phototrophic biomass using light energy include, without limitation, tanks, ponds, troughs, ditches, pools, pipes, tubes, canals, and channels. Such photobioreactors may be either open, closed, partially closed, covered, or partially covered. In some embodiments, for example, the photobioreactor 12 is a pond, and the pond is open, in which case the pond is susceptible to uncontrolled receiving of materials and light energy from the immediate environments. In other embodiments, for example, the photobioreactor 12 is a covered pond or a partially covered pond, in which case the receiving of materials from the immediate environment is at least partially interfered with. The photobioreactor 12 includes the reaction zone 10 which includes the reaction mixture. In some embodiments, the photobioreactor 12 is configured to receive a supply of phototrophic reagents (and, in some of these embodiments, optionally, supplemental nutrients), and is also configured to effect discharge of phototrophic biomass which is grown within the reaction zone 10. In this respect, in some embodiments, the photobioreactor 12 includes one or more inlets for receiving the supply of phototrophic reagents and supplemental nutrients, and also includes one or more outlets for effecting the recovery or harvesting of biomass which is grown within the reaction zone 10. In some embodiments, for example, one or more of the inlets are configured to be temporarily sealed for periodic or intermittent time intervals. In some embodiments, for example, one or more of the outlets are configured to be temporarily sealed or substantially sealed for periodic or intermittent time intervals. The photobioreactor 12 is configured to contain the reaction mixture which is operative for effecting photosynthesis upon exposure to photosynthetically active light radiation. The photobioreactor 12 is also configured so as to establish photosynthetically active light radiation (for example, a light of a wavelength between about 400-700 nm, which can be emitted by the sun or another light source) within the photobioreactor 12 for exposing the phototrophic biomass. The exposing of the reaction mixture to the photosynthetically active light radiation effects photosynthesis and growth of the phototrophic biomass. In some embodiments, for example, the established light radiation is provided by an artificial light source 14 disposed within the photobioreactor 12. For example, suitable artificial lights sources include submersible fiber optics or light guides, light-emitting diodes (“LEDs”), LED strips and fluorescent lights. Any LED strips known in the art can be adapted for use in the photobioreactor 12. In the case of the submersible LEDs, in some embodiments, for example, energy sources include alternative energy sources, such as wind, photovoltaic cells, fuel cells, etc. to supply electricity to the LEDs. Fluorescent lights, external or internal to the photobioreactor 12, can be used as a back-up system. In some embodiments, for example, the established light is derived from a natural light source 16 which has been transmitted from externally of the photobioreactor 12 and through a transmission component. In some embodiments, for example, the transmission component is a portion of a containment structure of the photobioreactor 12 which is at least partially transparent to the photosynthetically active light radiation, and which is configured to provide for transmission of such light to the reaction zone 10 for receiving by the phototrophic biomass. In some embodiments, for example, natural light is received by a solar collector, filtered with selective wavelength filters, and then transmitted to the reaction zone 10 with fiber optic material or with a light guide. In some embodiments, for example, both natural and artificial lights sources are provided for effecting establishment of the photosyntetically active light radiation within the photobioreactor 12.

“Aqueous medium” is an environment that includes water. In some embodiments, for example, the aqueous medium also includes sufficient nutrients to facilitate viability and growth of the phototrophic biomass. In some embodiments, for example, supplemental nutrients may be included such as one of, or both of, NO_{X} and SO_{X}. Suitable aqueous media are discussed in detail in: Rogers, L. J. and Gallon J. R. “Biochemistry of the Algae and Cyanobacteria,” Clarendon Press Oxford, 1988; Burlew, John S. “Algal Culture: From Laboratory to Pilot Plant.” Carnegie Institution of Washington Publication 600. Washington, D.C., 1961 (hereinafter “Burlew 1961”); and Round, F. E. The Biology of the Algae. St Martin's Press, New York, 1965; each of which is incorporated herein by reference). A suitable supplemental nutrient composition, known as “Bold's Basal Medium”, is described in Bold, H. C. 1949, The morphology of Chlamydomonas chlamydogama sp. nov. Bull. Torrey Bot. Club. 76: 101-8 (see also Bischoff, H. W. and Bold, H. C. 1963. Phycological Studies IV. Some soil algae from Enchanted Rock and related algal species, Univ. Texas Publ. 6318: 1-95, and Stein, J. (ED.) Handbook of Phycological Methods, Culture methods and growth measurements, Cambridge University Press, pp. 7-24).

Carbon dioxide-comprising exhaust material 14 is produced by a carbon dioxide-comprising gaseous exhaust material producing process 16. At least a fraction of the produced carbon dioxide-comprising exhaust material 14 is supplied to the respective reaction zone 10 of any one of the photobioreactors 12 to effect growth of the phototrophic biomass.

In some embodiments, for example, the carbon dioxide-comprising gaseous exhaust material 14 includes a carbon dioxide concentration of at least two (2) volume % based on the total volume of the carbon dioxide-comprising gaseous exhaust material 14. In some embodiments, for example, the carbon dioxide-comprising gaseous exhaust material 14 includes a carbon dioxide concentration of at least four (4) volume % based on the total volume of the carbon dioxide-comprising gaseous exhaust material 14. In some embodiments, for example, the gaseous exhaust material reaction 14 also includes one or more of N₂, CO₂, H₂O, O₂, NO_{R}, SO_{X}, CO, volatile organic compounds (such as those from unconsumed fuels) heavy metals, particulate matter, and ash. In some embodiments, for example, the carbon dioxide-comprising gaseous exhaust material 14 includes 30 to 60 volume % N₂, 5 to 25 volume % O₂, 2 to 50 volume % CO₂, and 0 to 30 volume % H₂O, based on the total volume of the carbon dioxide-comprising gaseous exhaust material 14. Other compounds may also be present, but usually in trace amounts (cumulatively, usually less than five (5) volume % based on the total volume of the carbon dioxide-comprising gaseous exhaust material 14).

In some embodiments, for example, the carbon dioxide-comprising gaseous exhaust material 14 includes one or more other materials, other than carbon dioxide, that are beneficial to the growth of the phototrophic biomass within the reaction zone 10. Materials within the gaseous exhaust material which are beneficial to the growth of the phototrophic biomass within the reaction zone 10 include SO_{X}, NO_{X}, and NH₃.

The carbon dioxide-comprising gaseous exhaust material producing process 16 includes any process which effects production and discharge of the carbon dioxide-comprising gaseous exhaust material 14. In some embodiments, for example, the carbon dioxide-comprising gaseous exhaust material producing process 16 is a combustion process. In some embodiments, for example, the combustion process is effected in a combustion facility. In some of these embodiments, for example, the combustion process effects combustion of a fossil fuel, such as coal, oil, or natural gas. For example, the combustion facility is any one of a fossil fuel-fired power plant, an industrial incineration facility, an industrial furnace, an industrial heater, or an internal combustion engine. In some embodiments, for example, the combustion facility is a cement kiln.

In some embodiments, for example, a supplemental nutrient supply 18 is supplied to the reaction zone 10 of any one of the photobioreactors 12. In some embodiments, for example, the supplemental nutrient supply 18 is effected by a pump, such as a dosing pump. In other embodiments, for example, the supplemental nutrient supply 18 is supplied manually to the reaction zone 10. Nutrients within the reaction zone 10 are processed or consumed by the phototrophic biomass, and it is desirable, in some circumstances, to replenish the processed or consumed nutrients. A suitable nutrient composition is “Bold's Basal Medium”, and this is described in Bold, H. C. 1949, The morphology of Chlamydomonas chlamydogama sp. nov. Bull. Torrey Bot. Club. 76: 101-8 (see also Bischoff, H. W. and Bold, H. C. 1963. Phycological Studies IV, Some soil algae from Enchanted Rock and related algal species, Univ. Texas Publ. 6318: 1-95, and Stein, J. (ED.) Handbook of Phycological Methods, Culture methods and growth measurements, Cambridge University Press, pp. 7-24). The supplemental nutrient supply 18 is supplied for supplementing the nutrients provided within the reaction zone, such as “Bold's Basal Medium”, or one or more dissolved components thereof. In this respect, in some embodiments, for example, the supplemental nutrient supply 18 includes “Bold's Basal Medium”. In some embodiments for example, the supplemental nutrient supply 18 includes one or more dissolved components of “Bold's Basal Medium”, such as NaNO₃, CaCl₂, MgSO₄, KH₂PO₄, NaCl, or other ones of its constituent dissolved components.

In some of these embodiments, the rate of supply of the supplemental nutrient supply 18 to the reaction zone 10 is controlled to align with a desired rate of growth of the phototrophic biomass in the reaction zone 10. In some embodiments, for example, regulation of nutrient addition is monitored by measuring any combination of pH, NO₃ concentration, and conductivity in the reaction zone 10.

In some embodiments, for example, a supply of the supplemental aqueous material supply 20 is effected to the reaction zone 10 of any one of the photobioreactors 12, so as to replenish water within the reaction zone 10 of the photobioreactor 12. In some embodiments, for example, and as further described below, the supplemental aqueous material supply 20 effects the discharge of product from the photobioreactor 12 by displacement. For example, the supplemental aqueous material supply 20 effects the discharge of product from the photobioreactor 12 as an overflow.

In some embodiments, for example, the supplemental aqueous material is water or substantially water. In some embodiments, for example, the supplemental aqueous material supply 20 includes aqueous material that has been separated from a discharged phototrophic biomass-comprising product 32 by a separator 50 (such as a centrifugal separator). In some embodiments, for example, the supplemental aqueous material supply 20 is derived from an independent source (i.e. a source other than the process), such as a municipal water supply.

In some embodiments, for example, the supplemental aqueous material supply 20 is supplied from a container that has collected aqueous material recovered from discharges from the process, such as aqueous material that has been separated from a discharged phototrophic biomass-comprising product.

In some embodiments, for example, the supplemental nutrient supply 18 is mixed with the supplemental aqueous material 20 in a mixing tank 24 to provide a nutrient-enriched supplemental aqueous material supply 22, and the nutrient-enriched supplemental aqueous material supply 22 is supplied to the reaction zone 10. In some embodiments, for example, the supplemental nutrient supply 18 is mixed with the supplemental aqueous material 20 within the container which has collected the discharged aqueous material. In some embodiments, for example, the supply of the nutrient-enriched supplemental aqueous material supply 18 is effected by a pump.

For each one of the photobioreactors 12, the reaction mixture disposed in the reaction zone 10 is exposed to photosynthetically active light radiation so as to effect photosynthesis. The photosynthesis effects growth of the phototrophic biomass.

In some embodiments, for example, the light radiation is characterized by a wavelength of between 400-700 nm. In some embodiments, for example, the light radiation is in the form of natural sunlight. In some embodiments, for example, the light radiation is provided by an artificial light source. In some embodiments, for example, light radiation includes natural sunlight and artificial light.

In some embodiments, for example, the intensity of the provided light is controlled so as to align with the desired growth rate of the phototrophic biomass in the reaction zone 10. In some embodiments, regulation of the intensity of the provided light is based on measurements of the growth rate of the phototrophic biomass in the reaction zone 10. In some embodiments, regulation of the intensity of the provided light is based on the molar rate of supply of carbon dioxide to the reaction zone 10.

In some embodiments, for example, the light is provided at pre-determined wavelengths, depending on the conditions of the reaction zone 10. Having said that, generally, the light is provided in a blue light source to red light source ratio of 1:4. This ratio varies depending on the phototrophic organism being used. As well, this ratio may vary when attempting to simulate daily cycles. For example, to simulate dawn or dusk, more red light is provided, and to simulate mid-day condition, more blue light is provided. Further, this ratio may be varied to simulate artificial recovery cycles by providing more blue light.

It has been found that blue light stimulates algae cells to rebuild internal structures that may become damaged after a period of significant growth, while red light promotes algae growth. Also, it has been found that omitting green light from the spectrum allows algae to continue growing in the reaction zone 10 even beyond what has previously been identified as its “saturation point” in water, so long as sufficient carbon dioxide and, in some embodiments, other nutrients, are supplied.

With respect to artificial light sources, for example, suitable artificial light source 14 include submersible fiber optics, light-emitting diodes, LED strips and fluorescent lights. Any LED strips known in the art can be adapted for use in the process. In the case of the submersible LEDs, the design includes the use of solar powered batteries to supply the electricity. In the case of the submersible LEDs, in some embodiments, for example, energy sources include alternative energy sources, such as wind, photovoltaic cells, fuel cells, etc. to supply electricity to the LEDs.

With respect to those embodiments where the reaction zone 10 is disposed in a photobioreactor 12 which includes a tank, in some of these embodiments, for example, the light energy is provided from a combination of sources, as follows. Natural light source in the form of solar light is captured though solar collectors and filtered with custom mirrors that effect the provision of light of desired wavelengths to the reaction zone 10. The filtered light from the solar collectors is then transmitted through light guides or fiber optic materials into the photobioreactor 12, where it becomes dispersed within the reaction zone 10. In some embodiments, in addition to solar light, the light tubes in the photobioreactor 12 contains high power LED arrays that can provide light at specific wavelengths to either complement solar light, as necessary, or to provide all of the necessary light to the reaction zone 10 during periods of darkness (for example, at night). In some embodiments, with respect to the light guides, for example, a transparent heat transfer medium (such as a glycol solution) is circulated through light guides within the photobioreactor 12 so as to regulate the temperature in the light guides and, in some circumstances, provide for the controlled dissipation of heat from the light guides and into the reaction zone 10. In some embodiments, for example, the LED power requirements can be predicted and, therefore, controlled, based on trends observed with respect to the produced carbon dioxide-comprising gaseous exhaust material 14, as these observed trends assist in predicting future growth rate of the phototrophic biomass.

In some embodiments, the exposing of the reaction mixture to photosynthetically active light radiation is effected while the supplying of the carbon dioxide-comprising gaseous exhaust material supply 15 is being effected.

In some embodiments, for example, the growth rate of the phototrophic biomass is dictated by the available carbon dioxide within the reaction zone 10. In turn, this defines the nutrient, water, and light intensity requirements to maximize phototrophic biomass growth rate. In some embodiments, for example, a controller, e.g. a computer-implemented system, is provided to be used to monitor and control the operation of the various components of the process disclosed herein, including lights, valves, sensors, blowers, fans, dampers, pumps, etc.

In some embodiments, for example, reaction zone product 30 is discharged from the reaction zone 10. The reaction zone product includes phototrophic biomass-comprising product 32. In some embodiments, for example, the phototrophic biomass-comprising product 32 includes at least a fraction of the contents of the reaction zone 10. In this respect, the discharge of the reaction zone product 30 effects harvesting of the phototrophic biomass 40.

In some embodiments, for example, the harvesting of the phototrophic biomass is effected by discharging the phototrophic biomass 32 from the reaction zone 10.

In some embodiments, for example, the discharging of the phototrophic biomass 32 from the reaction zone 10 is effected by displacement. In some of these embodiments, for example, the displacement is effected by supplying supplemental aqueous material supply 20 to the reaction zone 10. In some of these embodiments, for example, the displacement is an overflow. In some embodiments, for example, the discharging of the phototrophic biomass 32 from the reaction zone 10 is effected by gravity. In some embodiments, for example, the discharging of the phototrophic biomass 32 from the reaction zone 10 is effected by a prime mover that is fluidly coupled to the reaction zone 10.

In one aspect, the method includes, while a carbon dioxide-comprising gaseous exhaust material producing process 16 is effecting production of the carbon dioxide-comprising gaseous exhaust material 14, supplying at least a fraction of the produced carbon dioxide-comprising gaseous exhaust material 14 to a respective reaction zone 10 of each one of the phototobioreactors 12, in succession, wherein the at least a fraction of the produced carbon dioxide-comprising gaseous exhaust material being supplied defines a carbon dioxide-comprising gaseous exhaust supply 15.

Supplying the carbon dioxide-comprising gaseous exhaust supply 15 to a respective reaction zone 10 of each one of the phototobioreactors 12, in succession, means that the carbon dioxide-comprising gaseous exhaust supply 15 is supplied to a respective reaction zone of one of the photobioreactors 12 over a time interval, and at the completion of the time interval, the supplying of the carbon dioxide-comprising gaseous exhaust supply 15 to the respective reaction zone 10 of the one of the phototobioreactors is suspended, and after such suspension of the supplying, supplying of the carbon dioxide-comprising gaseous exhaust supply 15 to the respective reaction zone 10 of another one of the phototobioreactors is effected over a same or different time interval, and at the completion of such time interval, the supplying of the carbon dioxide-comprising gaseous exhaust supply 15 to the respective reaction zone 10 of the another one of the phototobioreactors is suspended. This continues until every one of the photobioreactors 12 is supplied by the carbon dioxide-comprising gaseous exhaust supply 15, independently, over a respective time interval. In some embodiments, for example, upon completion of the supplying of each one of the photobioreactors, in succession, by the carbon dioxide-comprising gaseous exhaust supply 15, a carbon dioxide-comprising exhaust supply cycle is thereby defined, and the carbon dioxide-comprising exhaust supply cycle is repeated at least once.

In some of these embodiments, for example, the carbon dioxide is being supplied by the carbon dioxide-comprising gaseous exhaust supply 15, at any given time during the process, to the reaction zone 10 of one of the photobioreactors 12. In some embodiments, for example, the supplying of the carbon dioxide-comprising gaseous exhaust supply 15 to a respective reaction zone 10 of each one of the phototobioreactors 12, in succession, independently, is effected over a respective time interval, and the supplying is continuous over that respective time interval. In some embodiments, for example, the supplying of the carbon dioxide-comprising gaseous exhaust supply 15 to a respective reaction zone 10 of each one of the phototobioreactors 12, in succession, independently, is effected over a respective time interval, and the supplying is semi-continuous or in intermittent pulses over that time interval.

In some embodiments, for example, for each one of the photobioreactors 12, growth of phototrophic biomass is being effected with the reaction zone 10.

In some embodiments, for example, the phototrophic biomass includes algae.

In some embodiments, for example, the supplying of the carbon dioxide-comprising gaseous exhaust supply 15 to a respective reaction zone 10 of each one of the phototobioreactors 12, in succession, is such that a carbon dioxide-comprising exhaust supply cycle is thereby defined. In some of these embodiments, for example, the carbon dioxide-comprising exhaust supply cycle is repeated at least once.

In some embodiments, for example, the carbon dioxide-comprising gaseous exhaust material supply 15 is defined by a fraction of the carbon dioxide-comprising gaseous exhaust material 14 being produced by the carbon dioxide-comprising gaseous exhaust material producing process 16, such that there is a remainder of the produced carbon dioxide-comprising gaseous exhaust material, and at least a fraction of the remainder of the produced carbon dioxide-comprising gaseous exhaust material 15 is being otherwise supplied to a respective reaction zone 10 of at least one of the photobioreactors 12. “Otherwise supplied” means that such fraction of the remainder is not included within the fraction that is being supplied by the produced carbon dioxide-comprising gaseous exhaust material 15 to the respective reaction zone 10 of each one of the photobioreactors 12, in succession.

In some embodiments, for example, the carbon dioxide-comprising gaseous exhaust material supply 15 being supplied is defined by the entire, or substantially the entire, carbon dioxide-comprising gaseous exhaust material 14 being produced by the carbon dioxide-comprising gaseous exhaust material producing process 16.

In some embodiments, for example, the supplying of the carbon dioxide-comprising gaseous exhaust supply 15 to a respective reaction zone 10 of each one of the phototobioreactors 12, in succession, independently, is effected over a respective time interval that is of a predetermined time duration.

In some embodiments, for example, the supplying of the carbon dioxide-comprising gaseous exhaust supply 15 to a respective reaction zone 10 of each one of the phototobioreactors 12, in succession, is effected over a respective time interval whose duration is the same or substantially the same.

In some embodiments, for example, while the pH, within the reaction zone 10 of the photobioreactor 12, which is being supplied by the carbon dioxide-comprising gaseous exhaust supply 15 (“the supplied photobioreactor”), is disposed above a predetermined low pH limit, the time interval over which the carbon dioxide-comprising gaseous exhaust supply 15 is being supplied to the supplied photobioreactor 12 is of a predetermined duration, and after the pH, within the reaction zone 10 of the supplied photobioreactor 12, becomes disposed below the predetermined low pH limit, the supplying of the carbon dioxide-comprising gaseous exhaust supply 15, to the reaction zone 10 of the supplied photobioreactor 12, becomes suspended such that the time interval, over which the carbon dioxide-comprising gaseous exhaust supply 15 is supplied to the reaction zone 10 of the supplied photobioreactor 12, is less than the predetermined duration. In some of these embodiments, for example, the suspension of the supplying of the carbon dioxide-comprising gaseous exhaust supply 15 to the supplied photobioreactor 12 is effected in response to detection of the pH, within the reaction zone 10 of the supplied photobioreactor 12, becoming disposed below the predetermined low pH limit.

In those embodiments where the supplying of the carbon dioxide-comprising gaseous exhaust supply 15 to a respective reaction zone 10 of each one of the phototobioreactors 12, in succession, is such that a carbon dioxide-comprising exhaust supply cycle is thereby defined, wherein the carbon dioxide-comprising exhaust supply cycle is repeated at least once, and after at least one cycle has been completed and a subsequent cycle has yet to begin or has been partially completed, upon the completion of the time interval, over which the supplying of the carbon dioxide-comprising gaseous exhaust supply 15 to the respective reaction zone 10 of any one of the photobioreactors 12 is effected, when the pH, within the reaction zone 10 of the following photobioreactor 12 to be supplied within the current cycle or the next cycle (if the photobioreactor 12, to whose reaction zone the supplying of the carbon dioxide-comprising gaseous exhaust supply 15 has been effected over the time interval which has been completed, is the last photobioreactor to be supplied within the current cycle, the following photobioreactor is the first photobioreactor to be supplied within the next cycle), becomes disposed below a predetermined low pH limit, the supplying of the carbon dioxide-comprising gaseous exhaust supply 15, to the reaction zone 10 of the following photobioreactor 12 is skipped for the current cycle, such that a bypassed photobioreactor is defined. In some embodiments, for example, the discharging of the gaseous photobioreactor exhaust 60 from within the bypassed photobioreactor is effected or continues to be effected.

In another aspect, the process for operating a plurality of photobioreactors includes, while a carbon dioxide-comprising gaseous exhaust material producing process 16 is effecting production of carbon dioxide-comprising gaseous exhaust material 14, and a carbon dioxide-comprising gaseous exhaust material supply 15, including at least a fraction of the produced carbon dioxide-comprising gaseous exhaust material 14, is supplied to a respective reaction zone 10 of one or more of the photobioreactors 12 (“the supplied photobioreactor(s)”), after the pH, within the reaction zone 10, of any one of the one or more supplied photobioreactor(s) 12, becomes disposed below a predetermined low pH limit, such that a low pH-disposed photobioreactor 12 is defined, at least a fraction of the carbon dioxide-comprising gaseous exhaust material supply 15, being supplied to the low pH-disposed photobioreactors, is diverted to a respective reaction zone 10 of each one of at least another one of the photobioreactors 12, for effecting supply of the diverted carbon dioxide-comprising gaseous exhaust material supply to the respective reaction zone 10 of each one of the at least another one of the photobioreactors 12. The at least a fraction of the carbon dioxide-comprising gaseous exhaust material supply 15 that is diverted defines the “diverted carbon dioxide-comprising gaseous exhaust material supply”. The diversion is such that there is a reduction in the molar rate of supply of carbon dioxide being supplied to the reaction zone of the low pH-disposed photobioreactor 12, and an increase in the molar rate of supply of carbon dioxide being supplied to the respective reaction zone of each one of the at least another one of the photobioreactors 12.

In some of these embodiments, for example, for each one of the photobioreactors 12, growth of phototrophic biomass is being effected with the reaction zone 10.

In some of these embodiments, for example, the phototrophic biomass includes algae.

In some embodiments, for example, the diverting is effected in response to detection of the pH, within the reaction zone 10 of the low pH-disposed photobioreactor 12, becoming disposed below the predetermined low pH limit.

In some embodiments, for example, the entire, or substantially the entire, carbon dioxide-comprising gaseous exhaust material supply 15, being supplied to the reaction zone 10 of the low pH-disposed photobioreactor 12, is diverted to a respective reaction zone 10 of at least another one of the photobioreactors 12, after the pH, within the respective reaction zone 10 of the low pH-disposed photobioreactor 12, becomes disposed below a predetermined low pH limit. In this respect, in such embodiments, for example, the diverting effects suspension of the supplying of the at least a fraction of the carbon dioxide-comprising gaseous exhaust material supply, to the reaction zone of the low pH-disposed photobioreactor. In some of these embodiments, for example, the diverting of the entire, or substantially the entire, carbon dioxide-comprising gaseous exhaust material supply 15, being supplied to the reaction zone 10 of the low pH-disposed photobioreactor 12, to the respective reaction zone 10 of each one of the at least another one of the photobioreactors 12, is effected in response to detection of the pH, within the reaction zone 10 of the low pH-disposed photobioreactor 12, becoming disposed below the predetermined low pH limit.

In some of these embodiments, for example, the respective reaction zone of each one of the at least another one of the photobioreactors 12, to which the diverted carbon dioxide-comprising gaseous exhaust material supply is diverted, includes a pH that is greater than the predetermined low pH.

In some embodiments, for example, the respective reaction zone 10 of each one of the at least another one of the photobioreactors 12, to which the diverted carbon dioxide-comprising gaseous exhaust material supply is diverted, includes a pH that is greater than or equal to the pH of the respective reaction zone 10 of every other one of the photobioreactors 12, other than the low pH-disposed photobioreactor 12.

In another aspect, while a carbon dioxide-comprising gaseous exhaust material producing process 16 is effecting production of carbon dioxide-comprising gaseous exhaust material 14, and a carbon dioxide-comprising gaseous exhaust material supply 15, including at least a fraction of the produced carbon dioxide-comprising gaseous exhaust material 14, is supplied to a respective reaction zone 10 of one or more photobioreactors 12 (“the supplied photobioreactor(s)”), after the pH, within the reaction zone 10, of any one of the one or more supplied photobioreactor(s) 12, becomes disposed above a predetermined maximum pH limit, such that a high pH-disposed photobioreactor 12 is defined, at least a fraction of the carbon dioxide-comprising gaseous exhaust material supply 15 being supplied to the respective reaction zone of each one of at least another one of the photobioreactors 12, whose reaction zone 10 includes a pH that is less than the pH within the reaction zone of the high pH-disposed photobioreactor, is diverted to the high pH-disposed photobioreactor 12, for effecting supply of the diverted carbon dioxide-comprising gaseous exhaust material supply to the reaction zone 10 of the high pH-disposed photobioreactor 12. In some of these embodiments, for example, the respective reaction zone of each one of the at least another one of the photobioreactors 12, from which the at least a fraction of the carbon dioxide-comprising gaseous exhaust material supply 15 is diverted to the reaction zone of the high pH-disposed photobioreactor 12, includes a pH that is less than or equal to the pH of the respective reaction zone 10 of every other one of the photobioreactors 12.

The diversion of the at least a fraction of the carbon dioxide-comprising gaseous exhaust material supply to the reaction zone 10 of the high pH-disposed photobioreactor 12, is such that there is a reduction in the molar rate of supply of carbon dioxide being supplied to the respective reaction zone of each one of the at least another one of the photobioreactors 12 (from which the at least a fraction of the carbon dioxide-comprising gaseous exhaust material supply is diverted), and an increase in the molar rate of supply of carbon dioxide being supplied to the reaction zone of the high pH-disposed photobioreactor 12.

With respect to those embodiments where pH within the reaction zone is sensed or detected, or where it is implicit that pH within the reaction zone 10 must be sensed or detected, a pH sensor is provided for sensing pH within the reaction zone 10. The pH sensor may be disposed for directly or indirectly sensing pH within the reaction zone 10. For example, in some embodiments, indirect sensing of pH within the reaction zone includes sensing of pH within the reaction zone product 60 being discharged from the reaction zone 10. The sensed pH is then transmitted to a controller. The controller compares the sensed pH to a predetermined value, and then determines what, if any, other action is to be taken, such as manipulating valves to reconfigure the supplying of the photobioreactors 12 by the carbon dioxide-comprising gaseous exhaust material supply 15.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments. Further, all of the claims are hereby incorporated by reference into the description of the preferred embodiments.

1. A process of operating a plurality of photobioreactors, comprising:
while a carbon dioxide-comprising gaseous exhaust material producing process is effecting production of the carbon dioxide-comprising gaseous exhaust material, supplying at least a fraction of the produced carbon dioxide-comprising gaseous exhaust material to a respective reaction zone of each one of the phototobioreactors, in succession, wherein the at least a fraction of the produced carbon dioxide-comprising gaseous exhaust material being supplied defines a carbon dioxide-comprising gaseous exhaust supply.

2. The process as claimed in claim 1;
wherein the supplying is such that a carbon dioxide-comprising exhaust supply cycle is thereby defined.

3. The process as claimed in claim 2;
wherein the carbon dioxide-comprising exhaust supply cycle is repeated at least once.

4. The process as claimed in claim 1;
wherein, for each one of the photobioreactors, the supplying of the carbon dioxide-comprising gaseous exhaust supply, to a respective reaction zone of a photobioreactor, is effected over a time interval that is of a predetermined time duration.

5. The process as claimed in claim 1;
while the pH, within the reaction zone of the photobioreactor, which is being supplied by the carbon dioxide-comprising gaseous exhaust supply, is disposed above a predetermined low pH limit, the time interval over which the carbon dioxide-comprising gaseous exhaust supply is being supplied to the supplied photobioreactor is of a predetermined duration, and after the pH, within the reaction zone of the supplied photobioreactor, becomes disposed below the predetermined low pH limit, the supplying of the carbon dioxide-comprising gaseous exhaust supply, to the reaction zone of the supplied photobioreactor, becomes suspended such that the time interval, over which the carbon dioxide-comprising gaseous exhaust supply is supplied to the reaction zone of the supplied photobioreactor, is less than the predetermined duration.

6. The process as claimed in claim 5;
wherein the suspension of the supplying of the carbon dioxide-comprising gaseous exhaust supply to the supplied photobioreactor is effected in response to detection of the pH, within the reaction zone of the supplied photobioreactor, becoming disposed below the predetermined low pH limit.

7. The process as claimed in claim 1;
wherein the supplying of the carbon dioxide-comprising gaseous exhaust supply to a respective reaction zone of each one of the phototobioreactors, in succession, independently, is effected over a respective time interval that is of a predetermined time duration.

8. The process as claimed in claim 1;
wherein the supplying of the carbon dioxide-comprising gaseous exhaust supply to a respective reaction zone of each one of the phototobioreactors, in succession, is effected over a respective time interval whose duration is the same or substantially the same.

9. The process as claimed in claim 3;
wherein, after at least one cycle has been completed and a subsequent cycle has yet to begin or has been partially completed, upon the completion of the time interval, over which the supplying of the carbon dioxide-comprising gaseous exhaust supply to the respective reaction zone of any one of the photobioreactors is effected, when the pH, within the reaction zone of the following photobioreactor to be supplied within the current cycle or the next cycle, becomes disposed below a predetermined low pH limit, the supplying of the carbon dioxide-comprising gaseous exhaust supply, to the reaction zone of the following photobioreactor is skipped for the current cycle

10. The process as claimed in claim 1;
wherein, for each one of the photobioreactors, growth of phototrophic biomass is being effected within the reaction zone by the supplied carbon dioxide.

11. The process as claimed in claim 1;
wherein the phototrophic biomass includes algae.

12. A process of operating a plurality of photobioreactors, comprising:
while a carbon dioxide-comprising gaseous exhaust material producing process is effecting production of carbon dioxide-comprising gaseous exhaust material, and a carbon dioxide-comprising gaseous exhaust material supply, including at least a fraction of the produced carbon dioxide-comprising gaseous exhaust material, is supplied to a respective reaction zone of one or more of the photobioreactors to thereby define one or more supplied photobioreactors, after the pH, within the reaction zone, of any one of the one or more supplied photobioreactors, becomes disposed below a predetermined low pH limit, such that a low pH-disposed photobioreactor is defined, at least a fraction of the carbon dioxide-comprising gaseous exhaust material supply, being supplied to the low pH-disposed photobioreactors, is diverted to a respective reaction zone of each one of at least another one of the photobioreactors, for effecting supply of the diverted carbon dioxide-comprising gaseous exhaust material supply to the respective reaction zone of each one of the at least another one of the photobioreactors.

13. The process as claimed in claim 12;
wherein the diverting is effected in response to detection of the pH, within the reaction zone of the low pH-disposed photobioreactor, becoming disposed below the predetermined low pH limit.

14. The process as claimed in claim 12;
wherein the diverting effects suspension of the supplying of the at least a fraction of the carbon dioxide-comprising gaseous exhaust material supply, to the reaction zone of the low pH-disposed photobioreactor.

15. The process as claimed in claim 12;
wherein the respective reaction zone of each one of the at least another one of the photobioreactors, to which the at least a fraction of the carbon dioxide-comprising gaseous exhaust material supply, previously being supplied to the reaction zone of the low pH-disposed photobioreactor, is diverted, includes a pH that is greater than the predetermined low pH.

16. The process as claimed in claim 12;
wherein the respective reaction zone of each one of the at least another one of the photobioreactors, to which the at least a fraction of the carbon dioxide-comprising gaseous exhaust material supply, previously being supplied to the reaction zone of the low pH-disposed photobioreactor, is diverted, includes a pH that is greater than or equal to the pH of the respective reaction zone of every other one of the photobioreactors, other than the low pH-disposed photobioreactor.

17. The process as claimed in claim 12;
wherein, for each one of the photobioreactors, growth of phototrophic biomass is being effected with the reaction zone.

18. The process as claimed in claim 12;
wherein the phototrophic biomass includes algae.

19. A process of operating a plurality of photobioreactors, comprising:
while a carbon dioxide-comprising gaseous exhaust material producing process is effecting production of carbon dioxide-comprising gaseous exhaust material, and a carbon dioxide-comprising gaseous exhaust material supply, including at least a fraction of the produced carbon dioxide-comprising gaseous exhaust material, is supplied to a respective reaction zone of one or more photobioreactors to thereby define one or more supplied photobioreactors, after the pH, within the reaction zone, of any one of the one or more supplied photobioreactors, becomes disposed above a predetermined maximum pH limit, such that a high pH-disposed photobioreactor is defined, at least a fraction of the carbon dioxide-comprising gaseous exhaust material supply being supplied to the respective reaction zone of each one of at least another one of the photobioreactors, whose reaction zone includes a pH that is less than the pH within the reaction zone of the high pH-disposed photobioreactor, is diverted to the high pH-disposed photobioreactor, for effecting supply of the diverted carbon dioxide-comprising gaseous exhaust material supply to the reaction zone of the high pH-disposed photobioreactor.

20. The process as claimed in claim 19;
wherein the respective reaction zone of each one of the at least another one of the photobioreactors, from which the at least a fraction of the carbon dioxide-comprising gaseous exhaust material supply is diverted to the reaction zone of the high pH-disposed photobioreactor, includes a pH that is less than or equal to the pH of the respective reaction zone of every other one of the photobioreactors.
